(19) United States
(12) Patent Application Publication
(10) Pub. No.: US 2025/0027077 A1
(43) Pub. Date: Jan. 23, 2025

(54) DEVICES AND METHODS FOR mtDNA EXTRACTION

(71) Applicant: Inso Biosciences, Inc., Ithaca, NY (US)

(72) Inventors: Adam Bisogni, Ithaca, NY (US); Irem Bastuzel, Ithaca, NY (US); Mohamed Rashed, Ithaca, NY (US); Azade Tahmsabei, Ithaca, NY (US); Harvey Tian, Ithaca, NY (US)

(21) Appl. No.: 18/778,205

(22) Filed: Jul. 19, 2024

Related U.S. Application Data
(60) Provisional application No. 63/514,943, filed on Jul. 21, 2023.

(51) Int. Cl. C12N 15/10; C12Q 1/6869

(52) U.S. Cl. CPC C12N 15/1093; C12N 15/1006; C12Q 1/6869

(57) ABSTRACT

Methods for extracting mtDNA include flowing a sample containing a cell into a microfluidic channel of a microfluidic device, capturing the cell on an array of microfeatures disposed within the microfluidic channel, lysing the captured cell and collecting lysate from the microfluidic device, sequencing nucleic acid from the lysate to obtain sequence data, and analyzing the sequence data to identify mtDNA sequences from the cell.

### BACKGROUND OF THE INVENTION

Intracellular organelles known as the mitochondria are sometimes called the powerhouse of the cell due to their role in generating a majority of cellular energy in the form of ATP. The mitochondria have their own mitochondrial DNA (mtDNA) genomes, independent from the cell's nuclear genome and mtDNA carries genes that are involved in ATP production. The animal mitochondrial genome is a closed circular double stranded DNA molecule of about 16 kilobases encoding 37 genes (coding for 13 proteins, 22 transfer RNAs, and 2 ribosomal RNAs). The mitochondrial genome is typically present in an abundant number of copies per cell.

It is understood that mutations in mtDNA are implicated in diseases such as neurodegenerative diseases, cancer and metabolic disorders. For example, more than 300 mtDNA protein coding and regulatory region variants have been reported to be associated with disease phenotypes. Additionally, while the behavior of the mitochondria in plants has evident importance for commercial agriculture, plant mtDNA has some unusual properties. For example, plant mtDNA exhibits only very low rates of synonymous substitution but very high rates of inversion and other internal rearrangements. In another example, certain plant mtDNAs actively transfer their genetic material to the nucleus. Plant mtDNAs are also remarkably active in horizontal gene transfer and are the only metazoan eukaryotic genomes known to transfer genes horizontally within a phylum.

### SUMMARY OF THE INVENTION

The invention provides devices and methods that are useful to extract nucleic acids with certain traits, such as mitochondrial genomes. In methods of the invention, a cell is captured in a microfluidic device and lysis is performed within the device. The lysis yields a lysate that includes mtDNA, potentially mixed with RNA, organelles, protein, membrane fragments, sugars, and nuclear DNA. The lysate is collected from the device and preferably subject to DNA purification to isolate a fraction of the lysate that contains DNA. That nucleic acid obtained from the lysate is then preferably sequenced to obtain sequence data. The sequence data is mapped to reference information to identify mtDNA sequences of the cell from the sample. Due to relatively high conservation across metazoan mitochondrial genomes, the reference mapping will reliably distinguish sequence reads of mitochondrial origin from sequence reads of nuclear origin. By having specifically identified the sequence reads of mitochondrial origin, methods of the invention provide mitochondrial-specific sequence information. In fact, those mitochondrial sequence reads may be subject to sequence assembly algorithms to reconstitute all or a substantial portion of the mitochondrial genome sequence for the cell.

When obtaining the DNA from the lysate (e.g., by a DNA purification), methods of the invention may also include a step for the selection or enrichment for mtDNA (e.g., size-selection) to provide a portion that is highly enriched for mtDNA, which mtDNA is thus available for storage or downstream analysis. For example, the enriched mtDNA may be simply sealed in a vessel or tube and/or frozen or submitted to a biological library. The enriched mtDNA may be subject to sequencing library preparation (e.g., fragmentation, adaptor ligation, and amplification) and optionally sequencing. The mtDNA may be subject to capture by probes, digital PCR detection, single-molecule sequencing, optical mapping, restriction digestion and restriction fragment length analysis, cloning, in vitro transcription, or any other analysis of interest.

Because methods of the invention are useful to identify a full, or substantial portion of, the sequence of a mitochondrial genome of a cell, the invention provides methods useful in a variety of clinical or agricultural analyses. For example, animal mtDNA can be enriched and analyzed to detect or diagnosis metabolic disorders, or crop mtDNA can be analyzed to detect mitochondrial genetic phenomena such as lateral gene transfer associated with commercially significant plant traits.

While illustrated here with examples specific to mitochondrial genomes, devices and methods of the invention may be used to capture any suitable target biological molecule from a sample. The invention may be particularly useful where the target biological molecule is highly conserved and/or where the target biological molecule exhibits uniformity in structure and/or size. For example, a selective lysis method or reagent may be used to preferentially lyse organelles over nuclei, and may be used to capture a variety of organelle-specific molecules such as nucleic acids or proteins from mitochondria, chloroplasts, and even intracellular parasitic or symbiotic organisms. Methods of the invention may also be suited to the capture of digestion-resistant nucleic acids such as covalently closed circles (e.g., mitochondrial genomes) or certain ribonucleoprotein-complexes such as ribosomes. Using size selection promotes capture and enrichment of targets of uniform size such as certain viral or symbiont genomes. Thus it will be seen that mtDNA is an example of an optimal target for methods and devices of the invention.

In certain aspects, the invention provides a method of analyzing mitochondrial DNA (mtDNA). The method includes flowing a sample containing a cell into a channel of a microfluidic device, capturing the cell on an array of microfeatures disposed within the channel, and lysing the captured cell. The method further includes collecting lysate from the microfluidic device, sequencing nucleic acid from the lysate to obtain sequence data, and analyzing the sequence data to identify mtDNA sequences from the cell. The sequence data may be obtained as a plurality of sequence reads and the analyzing step may include mapping the sequence reads to genomic reference data to identify sequence reads arising from a mitochondrial genome. Specifically, the analyzing step may include mapping sequence reads to a genomic database such as GenBank or the European Molecular Biology Laboratory (EMBL) to identify mitochondrial sequence reads. Mapping may be performed using a tool such as the Basic Local Alignment Search Tool.

In some embodiments, the nucleic acid comprises DNA and the method further includes, prior to the sequencing step, isolating the DNA from the lysate. The isolation step may be performed using a DNA isolation kit or column. The method may further include performing a purification or size selection on the lysate (e.g., before and/or after any DNA isolation step) to provide a fraction of the lysate enriched specifically for mtDNA. In embodiments where mtDNA is specifically purified or size selected into on fraction of the sample, the method may also include collecting nuclear genomic DNA and/or RNA (e.g., as another fraction from the purification or size selection step) into tubes or vessels, separate from the fraction of the lysate enriched for mtDNA. The method may include: (i) creating a sequencing library from the genomic DNA and/or RNA, and/or (ii) sequencing from the genomic DNA and/or RNA.

In certain embodiments, the sequencing step includes fragmenting the nucleic acid from the lysate to yield mtDNA fragments, ligating adaptors to the mtDNA fragments to produce adaptor-ligated fragments, and amplifying the adaptor-ligated fragments to make an mtDNA sequencing library. The method may include collecting the mtDNA sequencing library in one more sample tubes and freezing and/or storing the sample tubes containing the mtDNA sequencing library, prior to the sequencing step. The method may include loading the mtDNA sequencing library onto a nucleic acid sequencing platform and sequencing the library.

In some embodiments, the method includes fragmenting the nucleic acid in the lysate while on the microfluidic device to promote release of fragmented nucleic acid from the array of microfeatures. Fragmenting may include adding reagents such as exonuclease or other chemical (e.g., acid) reagents to the microfluidic device and/or mechanically fragmenting, e.g., by sonicating the microfluidic device to promote fragmentation of the nucleic acid while captured on the array of microfeatures.

The lysing step may include flowing a lysis agent (e.g., that includes a detergent and a chaotropic agent) into the microfluidic device to break or rupture at least a cell membrane and a mitochondrial membrane. Certain lysis agents or compositions of the disclosure may preferentially lyse cell and/or mitochondrial membranes over nuclear membrane.

After sequencing, the method includes analyzing the sequence data to identify mtDNA sequences from the cell. The method may include assembling mtDNA sequence reads to determine a sequence of the mitochondrial genome of the cell. For example, after a subset of the sequence reads are identified as mitochondrial (e.g., by mapping to a reference to by BLAST), that subset may be assembled together, e.g., by de novo sequence assembly, to determine a substantial portion (e.g., at least 30%, at least half, at least 80 to 90%, or all) of a mitochondrial genome. Homologous mitochondrial genomes from within a cell are likely to be substantially identical and also abundant from within the cell. Sequence reads from diverse mitochondria from within one cell may be assembled together as if from a single genomic origin. Accordingly, using the disclosed method with high-throughput, next-generation sequencing (NGS) instruments such as those from Illumina, mapping to identify mitochondrial sequence reads, and performing de novo assembly of those reads provides a mechanism for identifying full or substantial portions of sequences of mitochondrial genomes. Due to high conservation among the 37 mitochondrial genes, de novo assembly of mitochondrial genomes by methods of the invention may be productive even for unknown organisms, or those organisms for which mitochondrial genomic sequences are not yet know or published.

In other aspects, the invention provides methods for extracting mtDNA from a biological sample containing a cell. The method comprises flowing a sample containing a cell into a microfluidic channel of a microfluidic device, capturing the cell on an array of microfeatures disposed within the microfluidic channel, selectively lysing a cell membrane and a mitochondrial membrane of the captured cell without lysing the nuclear membrane to release mtDNA, eluting the mtDNA from the microfluidic device, and collecting the mtDNA. The selective lysis step may involve flowing selective lysis reagents into the device, which reagents may preferably include a detergent and a chaotropic salt that disrupt the cellular and mitochondrial membranes without rupturing any nuclear membrane. After the lysis step, lysate that will elute from the device includes mtDNA as well as membrane fragments, proteins, ribosomes and other RNA. That lysate is eluted as eluate from the device. Cellular nuclei remain intact and captured within the device. Note that those captured nuclei are available for a further, subsequent lysis and elution step to capture nuclear genomic material such as DNA and proteins. The initial eluate is collected and may optionally be further enriched for mtDNA, e.g., by size selection. A simple, optional size selection step such as running the eluate through a chromatography column and capturing a specific fraction allows one to specifically collect a portion of the original sample now significantly enriched for mtDNA.

Related aspects provide devices for isolating mtDNA. Devices of the invention may include a substrate or support comprising at least one inlet for receiving a sample containing a cell, at least one outlet, and at least one microfluidic channel extending from the inlet to the outlet. The microfluidic channel preferably includes an array of microfeatures and the device is provided with a selective lysis reagent to selectively lyse mitochondrial membranes of a cell while keeping the nuclear membrane intact. Methods and devices of the invention may be used to extract nucleic acids from other parts and organelles of the cells such as nucleic acid from the cytosol, chloroplast, nucleus etc. Various types of DNA extraction techniques may be used with methods and devices of the invention. For example, mitochondrial DNA can be extracted with the help of chemical agents such as detergents, enzymes, surfactants, and/or chaotropic salts as well as physical mechanisms such as sonication, acoustic waves, heat etc.

Methods of the invention may include sample preparation with and/or analysis of the extracted mtDNA. For example, extracted mtDNA may be subject to fragmentation, adaptor-ligation, and amplification to produce amplicons. Amplicons carry information from mitochondrial genomes and may be ascended to a biological library, or the amplicons may be sequenced to determine genomic sequences that are ascended to a genomic library.

The present disclosure relates to extracting or isolating certain DNA molecules such as the circularized DNAs that constitute the mitochondrial genome.

### BRIEF DESCRIPTION OF DRAWINGS

FIG. 1 diagrams a method for extracting mtDNA a sample.

FIG. 2 shows a microfluidic device for extracting mtDNA.

FIG. 3 is a photo micrograph of a device of the invention.

FIG. 4 shows parts of a device with first and second arrays of microfeatures.

FIG. 5 shows an array of microfeatures with substantially triangular cross-sections.

FIG. 6 shows an array of microfeatures with substantially rectangular cross-sections.

### DETAILED DESCRIPTION OF THE INVENTION

The present invention generally relates to methods, devices, and systems for the extraction, isolation, and analysis of target biological material having certain characteristics such as being membrane-bound but outside of the nucleus, uniform in size, and unavailable for digestion by exonuclease. Methods of the invention make use of selective lysis steps and reagents that do not lyse nuclear membranes while lysing other membranes including cellular membranes, organelles, and endoplasmic reticulum. In particular, methods of the invention make use of a microfluidic device that includes an array of microfeatures such as micropillars in a microchannel to capture one or more cells from a sample and old the cell at the array during the selective lysis. In fact, some devices may have an array of microfeatures with at least two sub-regions (or a channel with first and second arrays of microfeatures) such that, after selective lysis, an intact nucleus may flow from the first sub-region but be captured and held at a second sub-region where the microfeatures have a smaller or tighter spacing or size.

The present invention thus provides for capturing cells from a sample and extracting target material such as mtDNA from the cells. The methods, devices, systems are effective to selectively lyse the mitochondria of the captured cells without lysing the nucleus. Further, the device and system of the present invention can be used, after extraction of mtDNA, to capture genomic DNA (e.g., by lysing the captured nucleus after the mtDNA is eluted and collected).

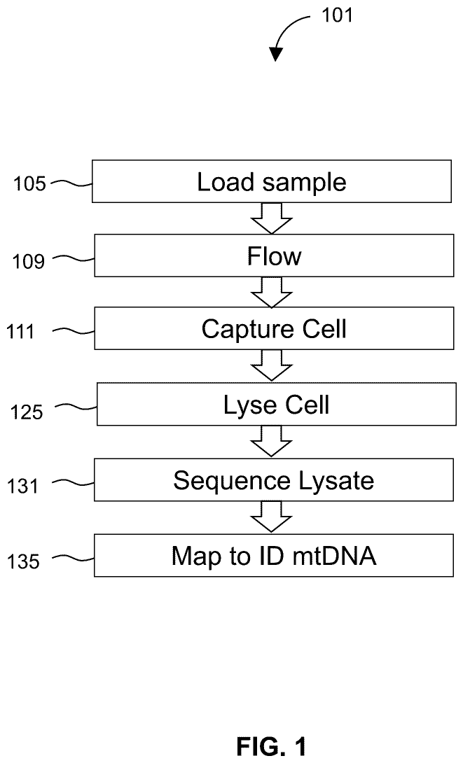

FIG. 1 diagrams a method 101 for extracting mtDNA from one or multiple cells contained in a biological sample. The method 101 includes loading 105 a sample containing a cell into a microfluidic device, e.g., into an input of the device and flowing 109 the sample from the input through a microfluidic cannel in the device. Any suitable sample may be subject to the method 101 including, for example, a blood or bodily fluid sample obtained from any suitable animal or subject. For example, the sample be a clinical sample that includes blood or bodily fluid from a human or veterinary patient. The sample may be from livestock, crops, or other plants, e.g., cultivated, farmed, or wild. The sample is loaded 105 into the channel via the input of the microfluidic device. The channel includes a plurality of microfeatures. As the sample flows 109 through the channel, a cell is captured 111 on the array of microfeatures disposed within the microfluidic channel.

The method 101 includes lysing 125 cells, thereby lysing cellular and mitochondrial membranes. The method further includes collecting lysate from the microfluidic device, sequencing 131 nucleic acid from the lysate to obtain sequence data, and analyzing the sequence data to identify mtDNA sequences from the cell, preferably by mapping 135 sequence reads to reference information. The sequence data may be obtained as a plurality of sequence reads and the analyzing step may include mapping 135 the sequence reads to genomic reference data to identify sequence reads arising from a mitochondrial genome. Specifically, the analyzing step may include mapping sequence reads to a genomic database such as GenBank or the European Molecular Biology Laboratory (EMBL) to identify mitochondrial sequence reads. Mapping may be performed using a tool such as the Basic Local Alignment Search Tool.

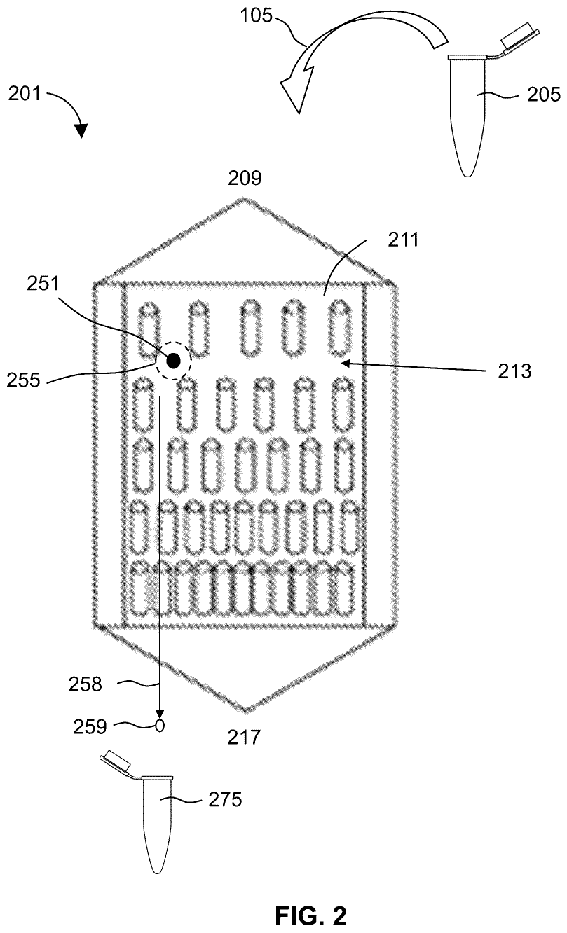

FIG. 2 shows a microfluidic device 201 for extracting mtDNA. The device 201 has a microfluidic channel 211 extending from an inlet 209 to an outlet 217. An array of microfeatures 213 (here shown as pillars) is disposed within the channel 211. When a sample 205 is loaded 105 into the inlet 209 and flowed through the channel 211, a cell 255 gets trapped on the array of microfeatures 213.

A lysis step 125 is performed to lyse or disrupt the membrane of the cell 255. The lysis process may include one or any combination of reagents, temperature, and mechanical activity. For example, mitochondrial and cellular membrane may be lysed by heating. Cell and mitochondrial membrane can also be lysed using mechanical agitation including but not limited to sonication and acoustic waves. In some embodiments, lysis 125 is performed using a selective lysis strategy that lyses cell or mitochondrial membranes preferably over nuclear membranes.

The lysis reagent flown in through the inlet, may include, without limitation, a detergent and a chaotropic salt. In particular, the detergents may be for example, Triton X-100, Tween 20. Chaotropic salts include but are not limited to n-butanol, ethanol, magnesium chloride, sodium dodecyl sulfate. Lysate 358 flows from the device 201 and is collected in a vessel 275. The lysate 258 includes nucleic acid including mtDNA 259.

Certain embodiments use a selective lysis reagents such as a detergent with a chaotropic salt. The selective lysis step 125 releases mtDNA 259 from the cell 255 (a selected lysis reagent with a detergent with a chaotropic salt may lyse cell and mitochondrial membranes preferentially over a nuclear membrane). The lysate may be collected 131 from the device 201, e.g., by washing an elution buffer or solution through the channel 211.

Preferably, the mtDNA 259 is collected 131 through the outlet 217 of the microfluidic device 201 into a collection vessel 275. The collection vessel 275 thus contains mtDNA e.g., potentially among other material such as RNA, organelles, proteins, or cell wall fragments.

The method may include isolating the DNA from the lysate. The isolation step may be performed using a DNA isolation kit or column. The method may optionally further include performing a purification or size selection on the isolated DNA, to provide a fraction of the lysate enriched specifically for mtDNA.

After nucleic acid is isolated or collected, the method may include fragmenting the nucleic acid from the lysate to yield mtDNA fragments, ligating adaptors to the mtDNA fragments to produce adaptor-ligated fragments, and amplifying the adaptor-ligated fragments to make an mtDNA sequencing library. For example, those steps may be performed to add first and second sequencing adaptors to amplicons formed in the library. Those adaptors may be the Y-adaptors referred to as P5 and P7 adaptors. At this stage, the protocol may be paused and the method may include collecting the mtDNA sequencing library in one more sample tubes and freezing and/or storing the sample tubes containing the mtDNA sequencing library, prior to the sequencing step. Preferably the method includes loading the mtDNA sequencing library onto a nucleic acid sequencing platform and sequencing the library to yield sequence data.

Any suitable sequencing platform may be used including, for example, sequencing using di-deoxy chain terminator known as Sanger sequencing, optionally on a capillary sequencing instrument. Sequencing may be performed using a long-read or single-molecule sequencing technology such as those from Oxford Nanopore or PacBio. Sequencing may be performed using a high-throughput NGS instrument such as those sold by Illumina or Ultima to produce a large number of short (e.g., 10 to 100 base) sequence reads.

Output from NGS sequencing platforms may be provided in any suitable format. For example, sequence reads maybe in a FASTA or FASTQ format, known in the art. The sequence data may be analyzed identify mtDNA sequences from the cell.

For example, the sequence data may be obtained as a plurality of sequence reads, e.g., short reads in a FASTQ or similar format. The sequence reads may be mapped to genomic reference data to identify sequence reads arising from a mitochondrial genome. Specifically, the analyzing step may include mapping sequence reads to a genomic database such as GenBank or the European Molecular Biology Laboratory (EMBL) to identify mitochondrial sequence reads. Mapping may be performed using a tool such as the Basic Local Alignment Search Tool. A practitioner may shell-script a tool to map reads to GenBank or used an existing bioinformatics pipeline such as BASESPACE by Illumina. For each read mapped to the reference, identifying information may read from the data base to label reads as mitochondrial or from some other source.

The method may include assembling mtDNA sequence reads to determine a sequence of the mitochondrial genome of the cell. For example, after a subset of the sequence reads are identified as mitochondrial (e.g., by mapping to a reference to by BLAST), that subset may be assembled together, e.g., by de novo sequence assembly, to determine a substantial portion (e.g., at least 30%, at least half, at least 80 to 90%, or all) of a mitochondrial genome. Sequence assembly may proceed using any of or any combination of a variety of suitable techniques such as those described in U.S. Pat. No. 8,209,130, incorporated by reference.

Homologous mitochondrial genomes from within a cell are likely to be substantially identical and also abundant from within the cell. Sequence reads from diverse mitochondria from within one cell may be assembled together as if from a single genomic origin. Accordingly, using the disclosed method with high-throughput, next-generation sequencing (NGS) instruments such as those from Illumina, mapping to identify mitochondrial sequence reads, and performing de novo assembly of those reads provides a mechanism for identifying full or substantial portions of sequences of mitochondrial genomes. Due to high conservation among the 37 mitochondrial genes, de novo assembly of mitochondrial genomes by methods of the invention may be productive even for unknown organisms, or those organisms.

Thus, for sample containing at least one cell that includes mitochondria, methods of the invention are useful to identify mitochondrial genomic sequence information for all, or a substantial amount of, the mitochondrial genome.

The microfluidic devices, methods, and systems of the present invention can be used to extract, isolate and analyze mtDNA from a variety of organisms including but not limited to bacteria, fungi, mammals such as humans, plants, marine organisms, algae etc. mtDNA can be extracted from a variety of cells such as stem cells, cancer cells, plant cells etc. Further, microfluidic devices, methods, and systems of the present invention can also be used to extract, isolate, and analyze other types of nucleic acid molecules from parts of the cells including but not limited to cytosol, chloroplast, nucleus etc. The microfluidic methods, systems, and devices of the present invention can be used to extract and isolate DNA from a sample that contains only a single cell or a population of cells, whether the number of cells in the population is small, medium, or large.

Methods of the present invention may further include extracting, isolating, and/or analyzing material other than mtDNA. For example, the methods of the present invention can be used for selectively extracting nucleic acid from other portions of the cells including but not limited to the cytosol, chloroplast, nucleus etc. In some embodiments, genomic DNA may be extracted, after eluting 131 mtDNA, by lysing the nucleus using lysis agents including but not limited to detergents, enzymes, surfactants, mechanical agents such as acoustic waves, heat etc. Once extracted, the genomic DNA from the cell may be temporarily immobilized within the micro-feature array of the microfluidic device. The immobilized genomic DNA may be maintained in elongated or non-elongated form when hydrodynamic force is applied to the microfluidic channel. The genomic DNA may then be released from the micro-feature array with the use of enzymatic digestion and collected from the outlet for further analysis.

While the method 101 is illustrated above with the device 201, other embodiments are within the scope of the disclosure. For example, device 201 is depicted with one array of microfeatures 213, shown as substantially cylindrical micropillars, in which spacing among the microfeatures decreases along the microchannel in a direction from the inlet 209 to the outlet 217. In other embodiments, the microfeatures may be present as two arrays (or two sub-regions of an array), in which a first, up-stream portion of the array has a first spacing and a second, downstream portion of the array has a second, tighter spacing.

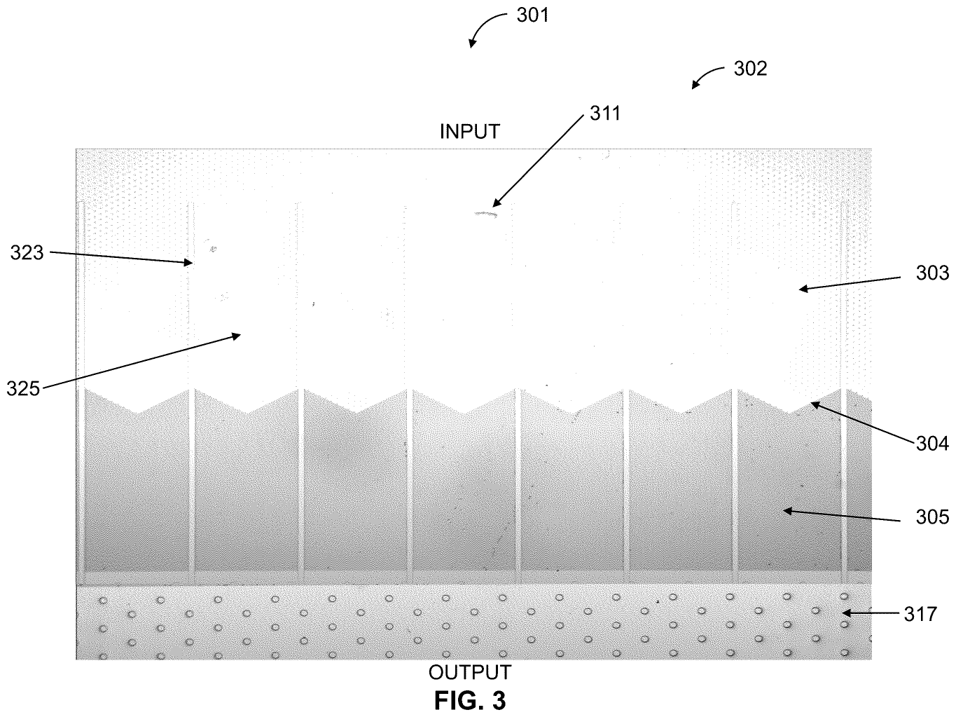

FIG. 3 is a photo micrograph of a device 301 that includes a first array of microfeatures 303 upstream of a second array of microfeatures 305 in a microfluidic channel 302. Preferably, the first array of microfeatures 303 is characterized by an inter-feature spacing suitable for capturing 111 a cell and the second array of microfeatures 305 has a second inter-feature spacing suitable for trapping a nucleus 251 while allowing mtDNA 259 to flow to an outlet of the device 301.

The device 301 is useful for isolating mtDNA from a cell. The device 301 includes a support 317 having an inlet for receiving the sample, an outlet for dispensing the flow-through or eluate, and a microfluidic channel 302 disposed within the support and extending from the inlet port to the outlet port. As shown, the microfluidic channel 302 occupies the entire width of the visible portion of the device 301. The device 301 may optionally include walls 323 placed in the microchannel 302 (e.g., at the valleys of the sawtooth boundary 304) to define parallel, adjacent lanes 325. The microfluidic channel includes a first array of microfeatures 303 and a second array of microfeatures 305.

In the reproduced photomicrograph, the first array of microfeatures 303 are micropillars that are visible (as dot-like marks in the picture). The second array of microfeatures 305 includes very fine micropillars that are small enough and close enough together that they appear as a uniform gray color across the middle of the figures. The first array of microfeatures 303 meets the second array of microfeatures 305 along a saw-tooth shaped boundary 304. There is no wall or other structure at the boundary. The boundary 304 is simply the span across the microchannel at which a fluid sample passes from the first array of microfeatures 303 to the second array of microfeatures 305.

The device 301 was manufactured from PDMS and the PDMS included some manufacturing imperfections 311 that are visible as some irregularly spaced dark marks in the photomicrograph but the imperfections 311 (dark marks) are not part of any array of microfeatures. The PDMS device 301 includes a surrounding supporting structure 317 that appears to include large pillars or columns (visible as about 70 circles in the bottom 10% of the photomicrograph). Those parts of the supporting structure 317 hold the device 301 together with appropriate dimensions for sample processing but do not participate directly in sample processing.

Other physical arrangements of features and embodiments of devices are within the scope of the disclosure.

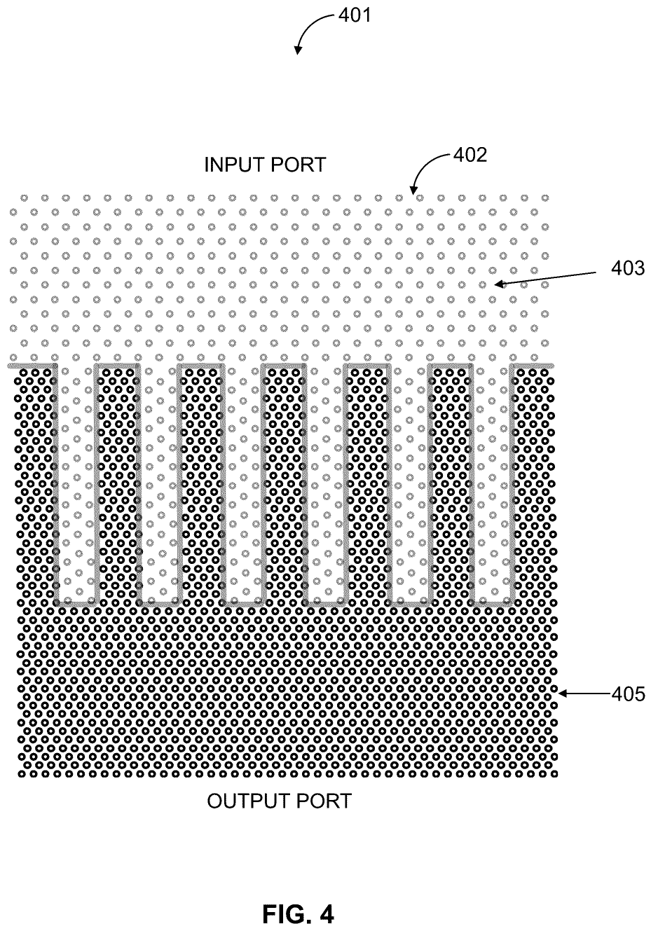

FIG. 4 shows parts of a device 401 that includes a first array of microfeatures 403 upstream of a second array of microfeatures 405 in a microfluidic channel 402. Preferably, the first array of microfeatures 403 is characterized by an inter-feature spacing suitable for capturing 111 a cell and the second array of microfeatures 405 has a second inter-feature spacing suitable for trapping a nucleus 251 while allowing mtDNA 259 to flow to an outlet of the device 401.

As discussed above, the features have been illustrated as micropillars or substantially cylindrical posts extending across a channel. However, any suitable physical microfeatures may be used that physically capture 111 a cell in a microchannel. The microfeatures may be micropillars, squares, triangles, rectangles, inverse/negative outline triangles, cross shapes, hexagons, diamonds, or any combination thereof.

FIG. 5 shows an example with an array of microfeatures with substantially triangular cross-sections.

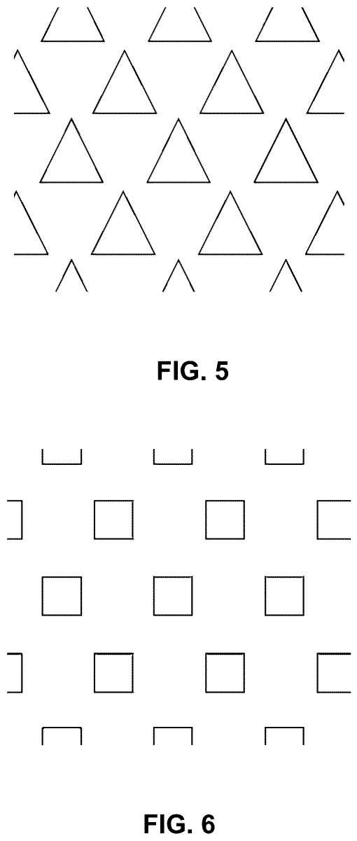

FIG. 6 shows an example with an array of microfeatures with substantially rectangular cross-sections. The microfeatures may be separated from each other by spacings between 2 micrometers and 150 micrometers. The array may include at least two sub-arrays that have (a) different microfeatures and/or (b) different microfeature spacing sizes. The at least 2 sub-arrays may be arranged in sequence along the channel in the direction of flow. Preferably, a first sub-array closer to the input port has microfeature spacing size that is larger than microfeature spacing size of a second sub-array that is closer to the output port. In certain embodiments comprising two microfeature spacing sizes (one interface between two microfeature spacing sizes), the microfeatures on the slide closest to the input port are spaced ten to a few hundred micrometers apart, and the microfeatures in region closest to the output port are spaced by less than hundred micrometers.

A system or kit of the invention includes a microfluidic device in which a microfluidic channel extends from an inlet to an outlet, an array of microfeatures disposed within the microfluidic channel, and selective lysis agent for use with the microfluidic device. When a cell is trapped on the array, the selective lysis agent may be introduced to the channel to selectively lyse mitochondrial membrane of the cell within the sample to release mtDNA, while keeping the nuclear membrane intact. The array microfeatures are spatially configured to immobilize cells allowing mtDNA to be eluted.

In one embodiment, the micro-feature array may include distinct regions for example, a first region and a second region. The first region may have cell-trapping spacing and the second region may have nuclei-trapping spacing among the microfeatures. In another embodiment, the micro-feature array may include only one region. In some embodiments, when the micro-feature array comprises two or more distinct regions, a first region has microfeatures spaced to entrap cells from the sample, while a downstream region has micro-features spaced to immobilize genomic DNA (gDNA; e.g., the device may include three regions for (i) cell capture, (ii) nuclei capture, and (iii) gDNA capture). In a particular embodiment, the micro-features of downstream regions are more closely spaced than the micro-features of upstream regions.

The present invention is not limited to this particular geometry, but instead the present invention contemplates a microfluidic channel that is the same width, greater width, smaller width, or variable width (smaller, greater, and/or the same width) as the inlet or outlet. Further, the present invention contemplates that an inflow channel or outflow channel can be of the same, substantially the same, or different widths and heights.

As described herein above, microfluidic devices of the invention may be formed on a support or substrate or base. Such a base or support portion can be made of material including but not limited to, polydimethylsiloxane (PDMS), polystyrene, epoxy, polymethylmethacrylate (PMMA), and silica. Microfeatures of an array and/or surfaces within a microchannel may be coated with at least one binding agent that has affinity to at least a portion of the surface of the at least one cell. In a particular embodiment, the at least one binding agent can be, without limitation, an antibody, an aptamer, or the like. Other binding agents known in the art that are effective to attract cells are also contemplated by the present invention.

In one embodiment, the system of the present invention can further include a hydrodynamic flow controller in functional communication with the outlet port and effective to generate hydrodynamic flow of a fluid from the inlet port to the outlet port. Suitable devices for use as a hydrodynamic flow controller of the present invention are known in the art and are contemplated for use in the present invention.

The present invention also relates to a method of making a microfluidic device for extracting and isolating DNA from at least one cell. The microfluidic device made by this method can also be used for analyzing the DNA that is extracted and isolated from the at least one cell. Methods of making the microfluidic device involves providing a support and forming within the support an inlet port for receiving a sample containing at least one cell, an outlet port for dispensing DNA isolated from the at least one cell, and a microfluidic channel disposed within the support and extending from the inlet to the outlet. The support can be made of a material including, but not limited to, polydimethylsiloxane (PDMS), polystyrene, epoxy, polymethylmethacrylate (PMMA), and silica. In one embodiment, the step of forming the support can involve using standard soft-lithography and/or injection molding.

The present invention also relates to a method of extracting or isolating mtDNA from a sample. Methods of the invention may optionally include performing sample preparation, library preparation, analysis, or sequencing of the mtDNA. Any suitable operation may be performed to analyze the mtDNA, including, without limitation, methods and tools involving fluorescence microscopy, optical microscopy, hybridization assays nucleic acid amplification, qPCR, and the like.

In certain embodiments, mtDNA that is eluted from a microfluidic device is further enriched (e.g., from cellular debris and other lysate) by a process such as size selection. For example, the lysate may be run through a column and only a fraction corresponding to covalently-closed dsDNA of about 15,000 to 17,000 bp may be collected and retained. Similarly, the lysate may be run out on a gel and a band corresponding to mitochondrial genomes may be excised.

Methods of the invention may optionally include a purification process at any suitable step in the method. Methods may involve purifying the mtDNA to remove proteins or other biomaterials bound or in contact with the DNA or released from the cell. In a particular embodiment, the purifying step can include contacting a proteinase buffer to the sample. The sample may be cleaned up with RNase or exonuclease (given that mitochondrial genomes are covalently closed).

Systems of the invention may include any apparatus useful for analyzing mtDNA such as a microscope, fluorescent microscope, hybridization array, mass spectrometer, DNA sequencing instrument, gel, or spectrophotometer. Systems or kits of the invention may include one or more reagents for use in any process of the disclosure. Suitable reagents may include, without limitation, a cell lysis agent, a DNA labeling agent, a restriction enzyme, and the like.

Methods for extracting mtDNA include flowing a sample containing a cell into a microfluidic channel of a microfluidic device, capturing the cell on an array of microfeatures disposed within the microfluidic channel, selectively lysing cellular and mitochondrial membranes without lysing a nuclear membrane to release mtDNA, eluting the mtDNA from the microfluidic device, and collecting the mtDNA.

In preferred embodiments, after the mtDNA is collected (and optionally size-selected), the mtDNA is subject to library preparation in a manner compatible with nucleic acid sequencing techniques. While library preparation may be specific for any of various sequencing techniques, exemplary techniques include Sanger sequencing, the next-generation sequencing (NGS) platform sold under the trademark ILLUMINA, and single-molecule sequencing. For prevailing NGS platforms, the mtDNA may be fragmented and to generate fragments. Sequencing adaptors such as Y-adaptors may be ligated to the fragments, which adaptors may include amplification primer binding sites, indexes, barcodes, and/or sequencing primer binding sites. The adaptor-ligated fragments may be amplified, e.g., by polymerase chain reaction (PCR) to generate sequencing-ready amplicons. Those amplicons constitute a sequencing library ready to be sequenced on an NGS instrument. The sequencing library may be collected into a suitable tube, such as a blood collection tube sold under the trademark VACUTAINER, a microcentrifuge tube sold under the trademark EPPENDORF, or a conical tube sold under the trademark FALCON tube. The tube may be sealed and stored or packaged, e.g., in a freezer or on dry ice, for later or analysis or for shipping. The sequencing library in that tube may be sequenced by a suitable sequencing technique.

### Example

This example describes one embodiment of the method and system of the present invention for use in, inter alia, extracting mtDNA from cells.

A polydimethylsiloxane (PDMS) microfluidic device for extraction and purification of genomic DNA from small cell populations was developed. Cells from blood sample may be trapped in a two-dimensional array of microfeatures in a microfluidic channel and lysed using sodium dodecyl sulphate such that mtDNA is extracted from the mitochondria of the cells while the nuclear membrane in the cells remained intact. The mtDNA may be flowed away from the microarray, eluted from the device, and collected for off-chip processing analysis, e.g., by size-selection, gel electrophoresis, or library preparation.

The entrapment and chemically-induced lysis of human cells in an array of microfeatures has been shown to be a highly-effective technique extracting DNA on a chip, Unlike conventional microchip-based extraction techniques, the presented physical capture mechanism does not depend on biochemical and electrostatic binding interactions between nucleic acids and functionalized surfaces. Here, by using a selective lysis that preserves the nuclear membrane intact, mtDNA is collected (genomic DNA may be separately eluted and collected by a subsequent process that includes lysing the nuclear membrane).

Methods and devices of the invention provide numerous advantages. Notably, the sample, the cell(s), and mtDNA all remain in aqueous solution throughout the entire process. At no stage does the sample or the mtDNA need to be centrifuged, spun dry, or otherwise dried. Compared to conventional methods, the extraction method of the present invention does not rely on DNA purification with magnetic microparticles or spin columns. The mtDNA is kept in aqueous solution at all stages of the method which avoids desiccation and breakage and provides the highest quality sample of substantially enriched and purified mtDNA.

1. A method of analyzing mitochondrial DNA (mtDNA), the method comprising: flowing a sample containing a cell into a channel of a microfluidic device; capturing the cell on an array of microfeatures disposed within the channel; lysing the captured cell and collecting lysate from the microfluidic device; sequencing nucleic acid from the lysate to obtain sequence data; and analyzing the sequence data to identify mtDNA sequences from the cell.

2. The method of claim 1, wherein the sequence data comprises a plurality of sequence reads and the analyzing step comprises mapping the sequence reads to genomic reference data to identify sequence reads arising from a mitochondrial genome.

3. The method of claim 1, wherein the nucleic acid comprises DNA and the method further includes, prior to the sequencing step, isolating the DNA from the lysate

4. The method of claim 3, wherein the isolation step is performed using a DNA isolation kit or column.

5. The method of claim 1, wherein the lysing step includes breaking at least a cell membrane and a mitochondrial membrane by flowing a lysis agent into the microfluidic device, wherein the lysis agent includes a detergent and a chaotropic agent.

6. The method of claim 1, wherein the analyzing step comprises mapping sequence reads of the sequence data to a genomic database to identify mitochondrial sequence reads.

7. The method of claim 6, wherein the mapping step uses a Basic Local Alignment Search Tool.

8. The method of claim 1, further comprising performing a purification or size selection on the lysate to provide a fraction of the lysate enriched for mtDNA.

9. The method of claim 8, further comprising collecting nuclear genomic DNA and/or RNA, from the purification or size selection step, in vessels or tubes separate from the fraction of the lysate enriched for mtDNA.

10. The method of claim 9, further comprising: (i) creating a sequencing library from the genomic DNA and/or RNA, and/or (ii) sequencing from the genomic DNA and/or RNA.

11. The method of claim 1, wherein the sequencing step further comprising: fragmenting the nucleic acid from the lysate to yield mtDNA fragments; ligating adaptors to the mtDNA fragments to produce adaptor-ligated fragments; and amplifying the adaptor-ligated fragments to make an mtDNA sequencing library.

12. The method of claim 9, further comprising collecting the mtDNA sequencing library in one more sample tubes and freezing and/or storing the sample tubes containing the mtDNA sequencing library, prior to the sequencing step.

13. The method of claim 1, further comprising fragmenting the nucleic acid in the lysate while on the microfluidic device to promote release of fragmented nucleic acid from the array of microfeatures.
